SEQUENCE LISTING (85 sequences)

SEQ ID NO: 1 - DNA, 663 bp, Meleagris gallopavo
atgtctgggaagccagttctgcactatcccaactcacgaggccgaatggaatcagtacggtggctgctagcagctgctggggttgagtttgaagaaaaatttctggaaaaaaaggaagatctccaaaagttacagtcggatggatccctgctgttccagcaagtgcccatggtggagatcgatgggatgaagttggtgcagaccagagccatcctcaactacatcgcagggaaatacaacctctacgggaaagacgtgaaagagagagccctaattgacatgtatgtggaaggaatggcagatctgtatgagttaatcatgatgaacgttgtccaaccagcagataaaaaggaggaacatcttgctaatgctttggacaaggccacaaacagatatttcccagtcttcgaaaagattttgaaccacgggcatgactttcttgttggcaacaagctgagcagagctgatgtgagtttactggaaaccattttagtggtggaagagcgcaagcctgatgcacttgcaaaatttcctctcttgcagagttttaaagcaagaataagcaatacccccaacatcaagaaattcctgcagcctggcagccagaggaaaccacctttacaggaaaaagatataccaagtctgatggcaattttccattga

SEQ ID NO: 2 - DNA, 666 bp, Meleagris gallopavo
atgtctgggaagccagttctgcactatgctaatatacgaggccgaatggaaccaataaggtggctactagcagctgctggagttgagtttgaagaaagaattctgaaaactaaggatgatctccaaaagttaaggacagatggatgcctgctgttccagcaagtgcccatggtggagatcgatgggatgaagatggtgcagaccagagccatcctcaactacatcgcagggaaatacaacctctacgggaaagacctgaaggagagagccctaattgacatgtatgtggaaggattggcagatctgtttgagttaatcatgtaccatgaattcaaacctgctaatgaaaaggagaaagatcttgcaaacattatggacaaggccacaaacagatacttaccagtctttgagaaggttttgaaagaccatgggcatgactttcttgttggcaacaagttgagcaaagcagatgtgagtttactggaaaacattttggggttggaagagttgaagcctgatgcacttgcaaaatttcctctcttgcagagttttaaagcaagaatgagcaatatccccagcatcaagaaattcctgcaacctggcagcgcaaagaaaccactgattcaggaaaaagatataccaagtgtgatagcaattttccactga

SEQ ID NO: 3 - DNA, 666 bp, Meleagris gallopavo
atgtctgggaagccagttctgcactatgtcagtgtacgcggccgaatggaaccagtgaagtggctactagcagctgctggagttgagtttgaagaaagatttctggaaacaaaagaagatctccaaaagttaagggcagatggatgcctgctgttccagcaagtgcccatggtggagatcgatgggatgaagttggtgcagaccagagccatcctcaactacatcgcagggaaatacaacctttacgggaaagacctgaaggagagagccctaattgacatgtatgtggaaggaatggcagatctgtatgatataatcatgaaccatgacttcaaacctgctaatgaaaaggagaaagatcttgcaaatattatggacaaggccacaaatagatacttaccagtctttgagaaggttttgaaagaccacgggcatgactttcttgttggcaacaagttgagcagagcagatgtgaatttactggaaactattttaatggtggaagagttcaagcctgatgcacttgcaaaatttcctctcttgcagagttttaaagccagaatgagcaatatccccagcatcaagaaattcctgcaacctggcagcccaaggaaaccacctctacaggaaaaagatataccaaacctgctggcaattttcagttga

SEQ ID NO: 4 - DNA, 749 bp, Meleagris gallopavo
aggcggagaaacctaagcttcactataccagaggaagggggaagatggagtcaatccggtggctgttagcagcagctggggttgaatttgaggaagaatttatagaaaaaaaagaagacctagaaaaattacgcaatgatggatccctgttgttccagcaagtgcccatggtggaaatcgatgggatgaagatggtgcagactagagccatcctctgctacatagcagggaaatacaatctctatgggaaagatctgaaggagagagcctggattgatatgtatgtggagggaacaacagacctgatgggaatgatcatggctctcccttttcaagcagccgatgtgaaagaaaagaatattgccttaatcactgaacgagctacaaccagatactttcctgtttatgaaaaggccttaaaagaccatggtcaggattatcttgttggcaacaagctaagctgggcagacatccacctgctggaagccattttaatgacagaagaacttaagtctgatatactctctgcattccctctgctacaggcttttaaaggaagaatgagcaacgtcccaacaatcaaaaaattcttgcagcctggcagccagaggaagccaccattagatgagaaatcgattgctaatgtgaggaaaatattcagtttctaatcatgtggcagctacagataacatgcccgtagtttattacctaagctgtgcctcttaagtgtaaactgcatagatctgtta

SEQ ID NO: 5 - DNA, 672 bp, Meleagris gallopavo
atgtcggagaagcccaggctcacctatatcaatggaagagggcgaatggagtccatccgatggctgctagctgcagctggagtggagtttgaagaaatttttctgcaaacaagagagcagttactgaagttaggccaagatggatccctgatgttccatcaattgccgctggttgagatcgacgggatgaagttggtgcagtgcagagccatcctcagctacatagcagggaaatacaatctctatgggaaagacttgaaggagagagccctgattgacatgtatgtggaaggaatatcagatctgatgcaattgattttgatgtttcctttctctccacctgaggcaaaggagaaaaatattgccacaatttcagagaaggcaacagagaggtacttccctgtctttgaaaaggtcttgaaacagcatggccaagactttcttgtgggaaaccgattcagctgggcagatgttcagctcatggaagccattttagcagtggaggagaaagtaccttctgtgctttctgggtttcctcagctgcaggcttttaaaaccaaaatgagcaacatgcctacaattaagaagttcctgcagcctggcagcccaagaaaacccccaccagatgaaaattatgtagcaactgtgatgaaaatttttaagctagactga

SEQ ID NO: 6 - DNA, 690 bp, Meleagris gallopavo
atggctgcaaaacctgtactctactactttgatggaagaggcaaaatggagtcgatccgctggctgctggctgcagctggggtcgagtttgaagaggtgtttttggaaacacgagagcagtatgagaaactcctgcaaagtggagtcctcatgttccagcaagtgcccatggtggagatcgatgggatgaagttggtgcagaccagagccatcctcaactacatcgcagggaaatacaacctctacgggaaagacctgaaggagagagccctgattgacatgtatgttgggggaacagatgaccttatgggcttcttattgagtttcccgttcttatcagctgaggataaggtgaaacaatgtgcctttgtagttcagaaggctacaagcaggtacttcccagcatatgaaaaggttttgaaagaccatggccaggactttcttgttggcaaccgtctcagctgggcagatattcatcttcttgaagccattttaatgctagaagaaaagaagtcagatgctctctcgggatttcctctgttacaggcatttaaaaaaaggataagcagcatccccacaatcaagaagttcctagcgcctggaagcaagagaaaacctatttctgatgataaatacgtggagactgtgaggagggttctccggatgtattatgatgtaaaaccaaattag

SEQ ID NO: 7 - DNA, 45 bp, Artificial Sequence
  Universal primer based on pBR322 purchased from Clontech Laboratories, Inc., Mountain View, CA.
ctaatacgactcactatagggcaagcagtggtatcaacgcagagt

SEQ ID NO: 8 - DNA, 22 bp, Artificial Sequence
  Universal primer based on pBR322 purchased from Clontech Laboratories, Inc., Mountain View, CA.
ctaatacgactcactatagggc

SEQ ID NO: 9 - DNA, 23 bp, Artificial Sequence
  Universal primer based on pBR322 purchased from Clontech Laboratories, Inc., Mountain View, CA.
aagcagtggtatcaacgcagagt

SEQ ID NO: 10 - DNA, 26 bp, Meleagris gallopavo
ctgtattactgttctgcagttaccca

SEQ ID NO: 11 - DNA, 26 bp, Meleagris gallopavo
cagagtagaattacattacgttgctg

SEQ ID NO: 12 - DNA, 25 bp, Meleagris gallopavo
caggcagatgtaagaggagcacttc

SEQ ID NO: 13 - DNA, 25 bp, Meleagris gallopavo
ttagaagacttcattgcgtggaagt

SEQ ID NO: 14 - DNA, 20 bp, turkey
ttttaagtttccccaggcag

SEQ ID NO: 15 - DNA, 27 bp, turkey
cacacactgtatcatactggaatttac

SEQ ID NO: 16 - DNA, 10 bp, Meleagris gallopavo
ggaaaggcaa

SEQ ID NO: 17 - DNA, 19 bp, Meleagris gallopavo
cattttttctttttttttt

SEQ ID NO: 18 - protein, 15 aa, Meleagris gallopavo
  misc_feature (1)..(1): Xaa can be any naturally occurring amino acid
XMEXXXWLLAAAGVE

SEQ ID NO: 19 - protein, 17 aa, Meleagris gallopavo
  misc_feature (5)..(5): Xaa can be any naturally occurring amino acid
YGKDXKERAXIDMYVXG

SEQ ID NO: 20 - protein, 15 aa, Meleagris gallopavo
  misc_feature (3)..(3): Xaa can be any naturally occurring amino acid
PVXEKVLKXHGXXXL

SEQ ID NO: 21 - protein, 18 aa, Meleagris gallopavo
  misc_feature (2)..(2): Xaa can be any naturally occurring amino acid
PXIKKFLXPGSXXKPXXX

SEQ ID NO: 22 - DNA, 28 bp, Artificial Sequence
  Universal primer based on pBR322 purchased from Clontech Laboratories, Inc., Mountain View, CA.
gcactgcaccaacttcatcccatcgatc

SEQ ID NO: 23 - DNA, 26 bp, Artificial Sequence
  Universal primer based on pBR322 purchased from Clontech Laboratories, Inc., Mountain View, CA.
gatgaacgtcgtccaaccagcagata

SEQ ID NO: 24 - DNA, 28 bp, Artificial Sequence
  Universal primer based on pBR322 purchased from Clontech Laboratories, Inc., Mountain View, CA.
cagcagggatccatcttggcataacttc

SEQ ID NO: 25 - DNA, 28 bp, Artificial Sequence
  Universal primer based on pBR322 purchased from Clontech Laboratories, Inc., Mountain View, CA.
tttagcggtggaagagtcgaagcctgat

SEQ ID NO: 26 - DNA, 26 bp, Artificial Sequence
  Universal primer based on pBR322 purchased from Clontech Laboratories, Inc., Mountain View, CA.
cacatcggctgcttgaaaagggagag

SEQ ID NO: 27 - DNA, 26 bp, Artificial Sequence
  Universal primer based on pBR322 purchased from Clontech Laboratories, Inc., Mountain View, CA.
ctctcccttttcaagcagccgatgtg

SEQ ID NO: 28 - DNA, 24 bp, Gallus gallus
gttccctccacatacatatcaatc

SEQ ID NO: 29 - DNA, 26 bp, Artificial Sequence
  Universal primer based on pBR322 purchased from Clontech Laboratories, Inc., Mountain View, CA.
ccatggtcaggattatcttgttggca

SEQ ID NO: 30 - DNA, 31 bp, Artificial Sequence
  Universal primer based on pBR322 purchased from Clontech Laboratories, Inc., Mountain View, CA.
gctacataattttcatctggtgggggttttc

SEQ ID NO: 31 - DNA, 27 bp, Artificial Sequence
  Universal primer based on pBR322 purchased from Clontech Laboratories, Inc., Mountain View, CA.
gccacaattgcagagaaggcaacagag

SEQ ID NO: 32 - DNA, 26 bp, Artificial Sequence
  Universal primer based on pBR322 purchased from Clontech Laboratories, Inc., Mountain View, CA.
tgcccagctgaatcggtttcccacaa

SEQ ID NO: 33 - DNA, 28 bp, Artificial Sequence
  Universal primer based on pBR322 purchased from Clontech Laboratories, Inc., Mountain View, CA.
agcagtggaggagaaagtgccttctgtg

SEQ ID NO: 34 - DNA, 27 bp, Gallus gallus
gccaacaagaaagtcctggccatggtc

SEQ ID NO: 35 - DNA, 28 bp, Gallus gallus
cccgttcttatcagctgaggataaggtg

SEQ ID NO: 36 - DNA, 30 bp, Gallus gallus
catgtcaatcagggctctctccttcaggtc

SEQ ID NO: 37 - DNA, 25 bp, Gallus gallus
aaggctacaagcaggtacttcccag

SEQ ID NO: 38 - DNA, 21 bp, Meleagris gallopavo
atgtctgggaagccagttctg

SEQ ID NO: 39 - DNA, 20 bp, Meleagris gallopavo
tcaatggaaaattgccatca

SEQ ID NO: 40 - DNA, 21 bp, Meleagris gallopavo
atgtctgggaagccagttctg

SEQ ID NO: 41 - DNA, 24 bp, Meleagris gallopavo
tcagtggaaaattgctatcacact

SEQ ID NO: 42 - DNA, 20 bp, Meleagris gallopavo
atgtctgggaagccagttct

SEQ ID NO: 43 - DNA, 21 bp, Meleagris gallopavo
tcaactgaaaattgccagcag

SEQ ID NO: 44 - DNA, 31 bp, Meleagris gallopavo
atggcggagaaacctaagcttcactatacca

SEQ ID NO: 45 - DNA, 35 bp, Meleagris gallopavo
taatgtgaaggaaaatattcagtttctaaggccgc

SEQ ID NO: 46 - DNA, 26 bp, Meleagris gallopavo
atgtcggagaagcccaggctcaccta

SEQ ID NO: 47 - DNA, 33 bp, Meleagris gallopavo
tcagtctagcttaaaaattttcatcacagttgc

SEQ ID NO: 48 - DNA, 27 bp, Meleagris gallopavo
atggctgcaaaacctgtactctactac

SEQ ID NO: 49 - DNA, 31 bp, Meleagris gallopavo
ctaatttggttttacatcataatacatccgg

SEQ ID NO: 50 - DNA, 24 bp, Meleagris gallopavo
ttaattaataatcagctgctttgc

SEQ ID NO: 51 - DNA, 19 bp, Meleagris gallopavo
gctagcagccagcgtactg

SEQ ID NO: 52 - DNA, 20 bp, Meleagris gallopavo
atggatccctgctgttccag

SEQ ID NO: 53 - DNA, 24 bp, Meleagris gallopavo
caaagactgggaaatatctgtttg

SEQ ID NO: 54 - DNA, 20 bp, Meleagris gallopavo
gcatcaggcttcgactcttc

SEQ ID NO: 55 - DNA, 22 bp, Meleagris gallopavo
gacatgtatgtggaaggactgg

SEQ ID NO: 56 - DNA, 18 bp, Meleagris gallopavo
cttcagctgccaggtttg

SEQ ID NO: 57 - DNA, 19 bp, Meleagris gallopavo
aaccccagctgctgctaac

SEQ ID NO: 58 - DNA, 19 bp, Meleagris gallopavo
gcgttatgcaaagcagagc

SEQ ID NO: 59 - DNA, 20 bp, Meleagris gallopavo
agcggatcgactccattttg

SEQ ID NO: 60 - DNA, 20 bp, Meleagris gallopavo
ctgcactatcccaactcacg

SEQ ID NO: 61 - DNA, 20 bp, Meleagris gallopavo
ctgcactatgctaatatacg

SEQ ID NO: 62 - DNA, 20 bp, Meleagris gallopavo
ctgcactatgtcagtgtacg

SEQ ID NO: 63 - DNA, 20 bp, Meleagris gallopavo
attcggcctcgtgagttggg

SEQ ID NO: 64 - DNA, 26 bp, Meleagris gallopavo
ggttccattcggcctcgtatattagc

SEQ ID NO: 65 - DNA, 20 bp, Meleagris gallopavo
attcggccgcgtacactgac

SEQ ID NO: 66 - DNA, 26 bp, Meleagris gallopavo
tgtaacttttggagatcttccttttt

SEQ ID NO: 67 - DNA, 25 bp, Meleagris gallopavo
tttggagatcatccttagttttcag

SEQ ID NO: 68 - DNA, 23 bp, Meleagris gallopavo
tggagatcttcttttgtttccag

SEQ ID NO: 69 - DNA, 20 bp, Meleagris gallopavo
ggttgtatttccctgcgatg

SEQ ID NO: 70 - DNA, 20 bp, Meleagris gallopavo
gtatgtggaaggaatggcag

SEQ ID NO: 71 - DNA, 21 bp, Meleagris gallopavo
ccattttagtggtggaagagc

SEQ ID NO: 72 - DNA, 20 bp, Meleagris gallopavo
ttttggggttggaagagttg

SEQ ID NO: 73 - DNA, 23 bp, Meleagris gallopavo
tttaatggtggaagagttcaagc

SEQ ID NO: 74 - DNA, 21 bp, Meleagris gallopavo
gccagaggaaaccacctttac

SEQ ID NO: 75 - DNA, 21 bp, Meleagris gallopavo
gcgcaaagaaaccactgattc

SEQ ID NO: 76 - DNA, 21 bp, Meleagris gallopavo
gcccaaggaaaccacctctac

SEQ ID NO: 77 - DNA, 21 bp, Meleagris gallopavo
ggaaaattgccatcagacttg

SEQ ID NO: 78 - DNA, 21 bp, Meleagris gallopavo
ggaaaattgctatcacacttg

SEQ ID NO: 79 - DNA, 21 bp, Meleagris gallopavo
tgaaaattgccagcaggtttg

SEQ ID NO: 80 - protein, 220 aa, Meleagris gallopavo
MSGKPVLHYPNSRGRMESVRWLLAAAGVEFEEKFLEKKEDLQKLQSDGSLLFQQVPMVEIDGMKLVQTRAILNYIAGKYNLYGKDVKERALIDMYVEGMADLYELIMMNVVQPADKKEEHLANALDKATNRYFPVFEKILNHGHDFLVGNKLSRADVSLLETILVVEERKPDALAKFPLLQSFKARISNTPNIKKFLQPGSQRKPPLQEKDIPSLMAIFH

SEQ ID NO: 81 - protein, 221 aa, Meleagris gallopavo
MSGKPVLHYANIRGRMEPIRWLLAAAGVEFEERILKTKDDLQKLRTDGCLLFQQVPMVEIDGMKMVQTRAILNYIAGKYNLYGKDLKERALIDMYVEGLADLFELIMYHEFKPANEKEKDLANIMDKATNRYLPVFEKVLKDHGHDFLVGNKLSKADVSLLENILGLEELKPDALAKFPLLQSFKARMSNIPSIKKFLQPGSAKKPLIQEKDIPSVIAIFH

SEQ ID NO: 82 - protein, 221 aa, Meleagris gallopavo
MSGKPVLHYVSVRGRMEPVKWLLAAAGVEFEERFLETKEDLQKLRADGCLLFQQVPMVEIDGMKLVQTRAILNYIAGKYNLYGKDLKERALIDMYVEGMADLYDIIMNHDFKPANEKEKDLANIMDKATNRYLPVFEKVLKDHGHDFLVGNKLSRADVNLLETILMVEEFKPDALAKFPLLQSFKARMSNIPSIKKFLQPGSPRKPPLQEKDIPNLLAIFS

SEQ ID NO: 83 - protein, 222 aa, Meleagris gallopavo
MAEKPKLHYTRGRGKMESIRWLLAAAGVEFEEEFIEKKEDLEKLRNDGSLLFQQVPMVEIDGMKMVQTRAILCYIAGKYNLYGKDLKERAWIDMYVEGTTDLMGMIMALPFQAADVKEKNIALITERATTRYFPVYEKALKDHGQDYLVGNKLSWADIHLLEAILMTEELKSDILSAFPLLQAFKGRMSNVPTIKKFLQPGSQRKPPLDEKSIANVRKIFSI

SEQ ID NO: 84 - protein, 223 aa, Meleagris gallopavo
MSEKPRLTYINGRGRMESIRWLLAAAGVEFEEIFLQTREQLLKLGQDGSLMFHQLPLVEIDGMKLVQCRAILSYIAGKYNLYGKDLKERALIDMYVEGISDLMQLILMFPFSPPEAKEKNIATIAEKATERYFPVFEKVLKQHGQDFLVGNRFSWADVQLMEAILAVEEKVPSVLSGFPQLQAFKTKMSNMPTIKKFLQPGSPRKPPPDENYVATVMKIFKLD

SEQ ID NO: 85 - protein, 229 aa, Meleagris gallopavo
MTAKPVLYYFDGRGKMESIRWLLAAAGVEFEEVFLETREQYEKLLQSGVLMFQQVPMVEIDGMKLVQTRAILNYIAGKYNLYGKDLKERALIDMYVGGTDDLMGFLLSFPFLSAEDKVKQCAFVVQKATSRYFPAYEKVLKDHGQDFLVGNRLSWADIHLLEAILMLEEKKSDALSGFPLLQAFKKRISSIPTIKKFLAPGSKRKPISDDKYVETVRRVLRMYYDVKPN

(19) United States
(12) Patent Application Publication
(10) Pub. No.: US 2013/0306000 A1
(43) Pub. Date: Nov. 21, 2013

(54) Aflaxtoxin Resistant Poultry

(71) Applicants: Roger A. Coulombe, Logan, UT (US); Kent Reed, Mounds View, MN (US)

(72) Inventors: Roger A. Coulombe, Logan, UT (US); Kent Reed, Mounds View, MN (US)

(73) Assignee: UTAH STATE UNIVERSITY, North Logan, UT (US)

(21) Appl. No.: 13/471,776

(22) Filed: May 15, 2012

(51) Int. Cl. C12Q 1/68; C07K 16/40; A01K 67/02; C40B 30/04; C07H 21/04; C40B 40/06; C40B 40/10

(52) U.S. Cl. 119/713; 506/16; 530/387.9; 506/18; 435/6.11; 506/9; 536/24.3

(57) ABSTRACT

The present disclosure includes methods of improving the quality of poultry and the efficiency of its production. Specifically, single nucleotide polymorphisms (SNPs) have been identified in signature domains and conserved residues found in α-class turkey Glutathione S-Transferase (GSTA) genes. These SNPS correlate with aflatoxin resistance in turkeys. Thus, methods of screening for the relevant SNPs using polynucleotides and anti-peptide antibodies, kits that include these tools, and methods of breeding aflatoxin resistant flocks are provided herein.

### STATEMENT REGARDING FEDERALLY SPONSORED RESEARCH

This invention was made with government support under grant 2002-35204-12294 awarded by the USDA-NIFA, and grant 2007-35205-17880 awarded by the USDA-NIFA. The government has certain rights in the invention.

### BACKGROUND

Glutathione S-transferases (GSTs: EC2.5.1.18) are a superfamily of multifunctional dimeric enzymes that catalyze the conjugation of glutathione (GSH) to electrophilic chemicals. In most animals and in humans, GSTs are the principal enzymes responsible for detoxifying the mycotoxin aflatoxin B1 (AFB1) and GST dysfunction is a known risk factor for susceptibility towards AFB1. Turkeys are among the most susceptible animals known to AFB1, which is a common contaminant of poultry feeds. The extreme susceptibility of turkeys is associated with hepatic GSTs unable to detoxify the highly reactive and electrophilic metabolite of AFB1, exo-AFB1-8,9-epoxide (AFRO). Susceptibility to AFB1 is problematic throughout the poultry industry.

### BRIEF SUMMARY

The present disclosure relates to DNA sequences within turkey glutathione S-transferase (GST) enzyme genes which represent single nucleotide polymorphisms (SNPs) that are associated with aflatoxin resistance in turkeys. Thus, methods and compositions of matter useful in the identification and/or promotion-of aflatoxin resistance in poultry are disclosed herein.

Also disclosed herein are comparative genomic approaches used to amplify and identify the α-class tGST genes (tGSTA1.1, tGSTA1.2, tGSTA1.3, tGSTA2, tGSTA3 and tGSTA4) from turkey liver. Further, conserved GST domains and four α-class signature motifs in turkey GSTs (with the exception of tGSTA1.1 which lacked one motif) that confirm the presence of hepatic α-class GSTs in turkeys are disclosed. Also disclosed herein are four signature motifs and conserved residues found in α-class tGSTs; the four signature motifs and conserved residues found in α-class tGSTs comprise (1) xMExxxWLLAAAGVE (SEQ ID NO: 18), (2) YGKDxKERAxIDMYVxG (SEQ ID NO: 19), (3) PVxEKVLKxHGxxxL (SEQ ID NO: 20), and (4) PxIKKFLXPGSxxKPxxx (SEQ ID NO: 21).

The isolation and sequencing of a bacterial artificial chromosome (BAC) clone containing the α-class GST gene cluster and turkey α-class GTS genes that genetically map to chromosome MGA2 with synteny between turkey and human α-class GSTs and flanking genes is also provided.

The present invention provides methods for improving the quality of poultry and the efficiency with which it may be produced. This is particularly true with regard to the production of turkey. Currently-bred domestic turkeys are especially susceptible to aflatoxin, a mycotoxin ubiquitous in most poultry feed stocks. We have identified specific SNPs within certain regions of turkey GSTA genes which are associated with aflatoxin resistance in the birds. GSTA enzymes in the liver break down aflatoxin and other toxins within the animal. The presence of these toxins in feed due to microorganisms that tend to be present in poultry feed makes aflatoxin resistance an important characteristic to have in birds in order to achieve efficient poultry production.

In one embodiment, the invention includes a method for determining aflatoxin susceptibility in animals, including poultry such as chickens or turkeys. This method first includes the step of obtaining a sample that contains genomic DNA or RNA from the animal. That sample may then be screened for the presence or absence of single nucleotide polymorphisms (SNPs) in an α-class glutathione S-transferase (GSTA) gene in the genomic DNA or RNA present in the sample. By then comparing the nucleotide sequences in the sample to sequences in a signature motif within GSTA nucleotide sequences that are associated with aflatoxin susceptibility, the user may determine whether the animal is susceptible to aflatoxin. Specifically, absence of all of the SNPs that we have identified in this invention indicates aflatoxin susceptibility.

According to the present invention, it has been determined that DNA sequences in specific GSTA genes correlate with aflatoxin resistance in animals. These GSTA genes include GSTA 1.1, GSTA 1.2, GSTA 1.3, GSTA2, GSTA3, or GSTA4 (SEQ ID NOS: 1-6). In particular, we demonstrate that the presence of SNPs identified in specific motifs of these GSTA genes is associated with aflatoxin resistance in turkeys (see Tables 6 and 7). These SNPs in the specific GSTA gene regions have been lost in domestic turkeys and this loss is associated with aflatoxin susceptibility in the turkeys. The association between the presence of SNPs in these regions of the GSTA genes is likely to exist in other genetically-similar species, including poultry such as chickens.

As described above, we demonstrate that wild turkeys have specific SNPs that associate with aflatoxin resistance. These include A₃₄, G₇₂, T₁₄₆, T₃₂₇, or T₅₆₉ in the GSTA1.1 nucleotide sequence (SEQ ID NO: 1), C₁₄₆, T₂₄₃, G₄₀₂, C₄₂₀, A₄₃₂, T₄₉₆ or T₆₀₆ of the GSTA 1.2 nucleotide sequence (SEQ ID NO: 2), C₄₈₇, T₅₁₄, or A₅₅₈ of the GTSA 1.3 nucleotide sequence (SEQ ID NO: 3), C₂₀₄, C₂₅₅, or A₆₃₆ of the GSTA2 nucleotide sequence (SEQ ID NO: 4), or T₂₁₉ or G₅₀₇ of the GSTA4 nucleotide sequence (SEQ ID NO: 6).

Our invention may be used by poultry producers to differentiate birds that are susceptible to aflatoxin from those that are resistant. This may be achieved by using isolated polynucleotides that include the regions that tend to include the SNPs within the GSTA genes (SEQ ID NOS: 1-6) described above, or the complement thereof. The isolated polynucleotides will include SNPs associated with aflatoxin resistance in turkeys. Specifically, the polynucleotides will each include one or more of the following GSTA sequences: T₃₄, A₇₂, C₁₄₆, C₃₂₇, or C₅₆₉ in the GSTA1.1 nucleotide sequence (SEQ ID NO: 1), G₁₄₆, C₂₄₃, A₄₀₂, G₄₂₀, G₄₃₂, G₄₉₆ or G₆₀₆ of the GSTA 1.2 nucleotide sequence (SEQ ID NO: 2), T₄₈₇, C₅₁₄, or C₅₅₈ of the GTSA1.3 nucleotide sequence (SEQ ID NO: 3), T₂₀₄, T₂₅₅, or G₆₃₆ of the GSTA2 nucleotide sequence (SEQ ID NO: 4), or C₂₁₉ or A₅₀₇ of the GSTA4 nucleotide sequence (SEQ ID NO: 6). Alternatively, a polynucleotide that represents the sequences in the domestic turkey in which the above SNPs have been lost may be used to identify aflatoxin susceptibility in animals. These sequences include A₃₄, G₇₂, T₁₄₆, T₃₂₇, or T₅₆₉ in the GSTA 1.1 nucleotide sequence (SEQ ID NO: 1), C₁₄₆, T₇₄₃, G₄₀₂, C₄₂₀, A₄₃₂, T₄₉₆ or T₆₀₆ of the GSTA1.2 nucleotide sequence (SEQ ID NO: 2), C₄₈₇, T₅₁₄, or A₅₅₈ of the GTSA1.3 nucleotide sequence (SEQ ID NO: 3), C₂₀₄, C₂₅₅, or A₆₃₆ of the GSTA2 nucleotide sequence (SEQ ID NO: 4), or T₂₁₉ or G₅₀₇ of the GSTA4 nucleotide sequence (SEQ ID NO: 6).

The polynucleotides described above may be incorporated into a kit which the user may conveniently use to assess the aflatoxin resistance or susceptibility of specific animals. The technique for using the kit may include labeling the polynucleotides so that a signal may be produced to detect those polynucleotides that have hybridized with the test subject's DNA, RNA, or DNA synthesized using the test subject's DNA or RNA as a template. One method of using these polynucleotides is to immobilize polynucleotides that include the sequences described above on a substrate. One of skill in the art will appreciate that polynucleotides may be labeled with a variety of methods including the use of radioisotopic, chemiluminescent, and colorimetric signals. The substrate may then be exposed to the test subject's DNA, RNA, or DNA synthesized using the test subject's RNA or DNA and given the opportunity to hybridize to the polynucleotides on the substrate. The test sample will hybridize to those polynucleotides which represent the complement to the sequence in the test sample. The presence or absence of the relevant SNPs can then be determined by detecting which polynucleotides hybridized to test sample nucleotides. Aflatoxin resistance or susceptibility in the test subject may then be determined according to the presence or absence of relevant SNPS. An alternate approach such as allele-specific PCR may also be employed to produce comparable results.

Some of the SNPs code for a different amino acid than those observed in domestic turkeys. In these situations, anti-peptide antibodies which differentially bind to peptides with or without the relevant SNPs could be used to identify aflatoxin resistance or susceptibility. Specifically, anti-peptide antibodies that bind to one or more of Ser₁₂, Ser₄₉, or Thr₁₉₀ of the GSTA1.1 peptide (SEQ ID NO: 80), Cys₄₉, Leu₁₄₀, or Gly₁₆₆ of the GSTA 1.2 peptide (SEQ ID NO: 81), or Pro₁₇₂ of GSTA1.3 peptide (SEQ ID NO: 82) could be used to identify aflatoxin resistance in subject animals.

Alternatively, anti-peptide antibodies that bind to the amino acid sequences that are produced in domestic turkeys could be used to detect aflatoxin susceptibility. These antibodies would include those that specifically bind to one or more of Thr₁₂, Phe₄₉, or Ile₁₉₀ of the GSTA 1.1 peptide (SEQ ID NO: 80), Ser₄₉, Phe₁₄₀, or Trp₁₆₆ of the GSTA 1.2 peptide (SEQ ID NO: 81), or Ser₁₇₂ of GSTA1.3 peptide (SEQ ID NO: 82). An assay using the antibodies could be developed in kit form in which the user detects the polypeptide that results from the presence or absence of the relevant SNPs when using a protein sample isolated from test subject animals. Like the polynucleotides, the antibodies could be immobilized to a substrate in an array and the isolated protein from the test subject allowed to interact with the antibodies on the substrate. Those antibodies which bind to the test subject's protein sample would indicate which amino acid sequence is present in the test subject's GSTA proteins.

The assays described to differentiate between animals that possess the SNPs from those that do not could be provided direct-to-consumer or, alternatively, could be conducted by a designated laboratory which would provide the testing as a service to poultry producers who send in a sample to be assayed.

Once animals that possess the relevant SNPs in their genome are identified using the techniques described above, it will be possible to generate aflatoxin resistant flocks from founder animals. Specifically, birds would be screened for the presence of the relevant SNPs using one of the techniques described above. Then birds found to possess the GSTA nucleotide sequences associated with aflatoxin resistance would serve as founder birds for breeding. The offspring from these founder birds that possess the SNPs in their GSTA genes that are associated with aflatoxin resistance would then be screened to identify birds homozygous for GSTA sequence associated with aflatoxin resistance. This F1 generation would then be cross-bred to create a flock that consists of birds that are homozygous for GSTA sequence associated with aflatoxin resistance and, consequently, an aflatoxin resistant flock. This process may be performed in a variety of poultry species including turkeys and chickens.

### BRIEF DESCRIPTION OF THE FIGURES

FIG. 1 shows the primers used to amplify GSTA fragments from turkey cDNA using 5′ and 3′ Rapid Amplification cDNA Ends (RACEs) (SEQ ID NOs: 22-37).

FIG. 2 shows sequence features of the turkey α-class GST cluster.

FIG. 3 shows an alignment of the amino acid sequences of tGSTA1.1 (SEQ ID NO: 80), tGSTA1.2 (SEQ ID NO: 81), tGSTA1.3 (SEQ ID NO: 82), tGSTA2 (SEQ ID NO: 83), tGSTA3 (SEQ ID NO: 84) and tGSTA4 (SEQ ID NO: 85).

### DETAILED DESCRIPTION

The present invention relates to the identification of SNPs in genes encoding hepatic GSTs in animals, and more specifically in poultry, which correlate with resistance to aflatoxin in the animal. More specifically, the SNPs identified herein allow testing of animals, such as commercially valuable poultry species, to identify individuals possessing hepatic GSTs able to detoxify aflatoxin and metabolites thereof, including, without limitation, the AFB1 metabolite exo-AFB1-8,9-epoxide (AFBO). Such individuals would be generally more commercially desirable, and could be selected for breeding to provide commercial strains of aflatoxin-resistant poultry, including turkeys and chickens.

Thus, in one embodiment of the present invention, a method for identifying aflatoxin resistant animals, such as poultry, is provided. In a more specific embodiment, methods for identifying aflatoxin resistant turkeys or chickens are provided. Throughout this specification, the terms “aflatoxin resistance” or variants thereof are used to denote the ability of an organism to detoxify the toxic AFB1 metabolite AFB1-8,9-epoxide (AFBO) via GSTs.

As explained briefly above, such methods are useful in identifying aflatoxin resistant individuals, and could thus be used in the promotion of aflatoxin resistance in commercial turkey flocks. The extreme sensitivity of turkeys is strongly associated with unresponsiveness of hepatic GSTs toward AFBO. Such turkey flocks would be more robust in general and result in stronger poultry strains, and potentially a healthier food product.

In these methods of these embodiments of the present invention, an organism is identified for testing, and a sample is obtained from the organism for analysis. The sample includes genomic DNA or RNA to allow proper detection of the SNPs of the invention which correlate with resistance to aflatoxin. Presence of the above-referenced SNPs, discussed in greater detail below, indicates aflatoxin resistance, while absence of the SNPs identified herein indicates lack of resistance to aflatoxin.

In another example, the present invention provides methods of providing poultry products with reduced levels of aflatoxin and aflatoxin-related toxins such as metabolites of aflatoxin. We have shown that while some turkey livers contain catalytically active GSTs, none of which possess affinity toward AFBO, a similar situation may exist in some humans where constitutively-expressed hepatic α-class GSTs have little or no AFBO detoxifying activity. By consuming poultry products with reduced aflatoxin and aflatoxin-related toxins, humans that express GSTs with reduced affinity toward AFBO would experience reduced toxicity.

Such methods would include the steps of identifying aflatoxin resistant poultry such as turkeys or chickens, as discussed herein, and either providing such resistant individuals for consumption and/or using such resistant individuals as breed stock to generate flocks of resistant animals for use as a human food source.

In another example of the above embodiments, there are provided tGST genes and genetic analyses useful in reducing aflatoxins, aflatoxin sensitivity, and aflatoxin-related toxins in poultry and poultry products. Despite the large impact of this mycotoxin to the poultry industry, little previous information has been available about the functional characteristics of avian GSTs, especially in the context of AFB1 detoxification. To our knowledge, this is the first study to fully amplify, identify, sequence and map turkey α-class GSTs. Consequently, we disclose the first genetic analyses which will identify aflatoxin sensitivity in avian species. These analyses may be used to identify aflatoxin sensitive birds and, thus, to develop aflatoxin resistant flocks.

Comparative genomic approaches were used to genetically map the α-class GST cluster to turkey chromosome MGA2 and the observed synteny between turkey and human α-class GSTs are reflected in annotation of turkey GSTAs. Comparative gene mapping is an effective tool for the study of genome evolution in phylogenetically distant species (turkey, chicken and human) that represent key stages in vertebrate evolution. The GST cluster provides insight into the origin and evolution of duplicated gene families. Identifying the complete sequence of the turkey α-class GSTs allowed identification of the SNPs of the present invention and may be used to identify other markers associated with AFB1 susceptibility and resistance in animals and in poultry species such as turkeys and chickens.

All six GSTAs identified herein were shown to contain two conserved GST domains: the GSH-binding site (G subsite) and the hydrophobic substrate-binding site (H subsite) which is subject to variation across the classes. The presence of four signature motifs in turkey GSTs (with the exception of tGSTA1.1 which lacked one motif) suggests the presence of intact GSTAs in turkey livers. While some GSTs share substrate specificities, there are distinct differences in substrate preference between subfamilies. Sequence similarity between classes is rather low, ranging between 20-30%. Certain detoxification enzymes such as GSTs are reported to be transcriptionally regulated through an enhancer known as the antioxidant response elements. Expression of the ABO metabolizing mGSTA3 regulated by the Nrf2 transcription factor through an antioxidant response element was reported in mice.

Based on our complete sequences of tGSTA (Genbank accession no. GQ254850), we have identified several putative transcription factors in the 5′-regulatory elements of these GSTs. The genomic approach discussed herein provides the framework necessary to identify genetic markers related to AFB1 susceptibility and resistance in turkeys with the ultimate goal of reintroducing AFB1-protective alleles into commercial populations.

### DEFINITIONS

tGST as used herein means turkey GST gene, and the terms “tGST” and “turkey GST” are used interchangeably.

Poultry as used herein means any avian species including farm animals but also nondomestic species of birds and those not commonly used as food animals.

Toxin as used herein means any naturally occurring or man-made substance that causes harm to an organism when said organism ingests, inhales, touches externally, or otherwise physically contacts the substance.

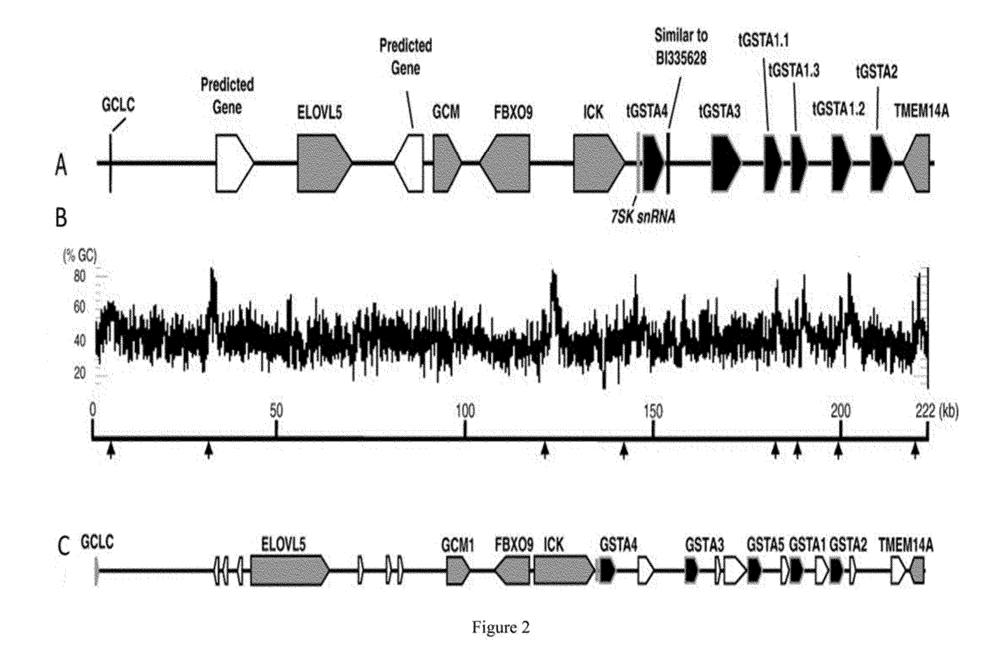

FIG. 2 shows sequence features of the turkey α-class GST cluster. FIG. 2A demonstrates genes and orientation as predicted within the turkey GST BAC. GST genes are indicated in black, and other genes with supporting EST data are indicated in gray. Genes predicted in silico are indicated in white.

FIG. 2B illustrates the GC content calculated by continuous 100 bp windows, with arrowheads used to denote the position of CpG islands. FIG. 2C provides the homologous human sequence (˜0.8 Mb) from 6p12.1-12.2, showing the position of predicted genes, which are coded as in FIG. 2A. Signature motifs and specific conserved residues of α-class tGSTs (shaded boxes) are indicated in (1) 15-29 aa (xMExxxWLLAAAGVE) (SEQ ID NO: 18), (2) 82-98 aa (YGKDxKERAxIDMYVxG) (SEQ ID NO: 19), (3) 134-148 aa (PVxEKVLKxHGxxxL) (SEQ ID NO: 20), and (4) 191-208 aa (PxIKKFLxPGSxxKPxxx) (SEQ ID NO: 21). A single amino acid (142 aa) is missing in tGSTA1.1 (2).

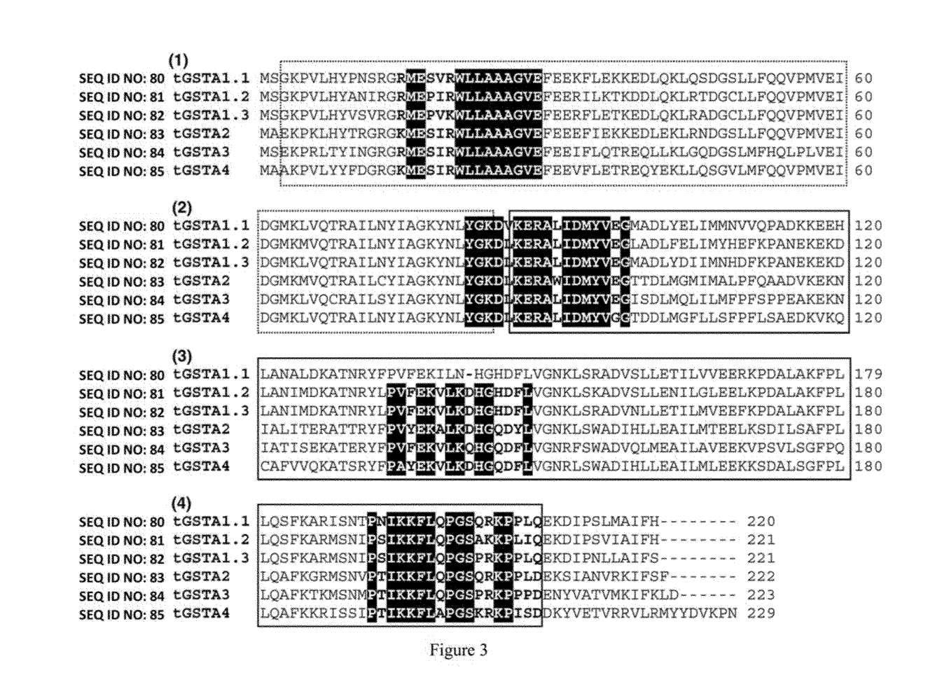

FIG. 3 provides an alignment of the amino acid sequences of tGSTA1.1 (SEQ ID NO: 80), tGSTA1.2 (SEQ ID NO: 81), tGSTA1.3 (SEQ ID NO: 82), tGSTA2 (SEQ ID NO: 83), tGSTA3 (SEQ ID NO: 84) and tGSTA4 (SEQ ID NO: 85), six GSTs obtained from turkey liver. The conserved N-terminal thioredoxin domain (amino acids 3-83) is outlined with a dotted line; and the conserved C-terminal conserved α-helical domain (amino acids 85-207) is outlined with a solid line.

### Materials and Methods

RNA Extraction and Rapid Amplification of cDNA Ends (RACE)

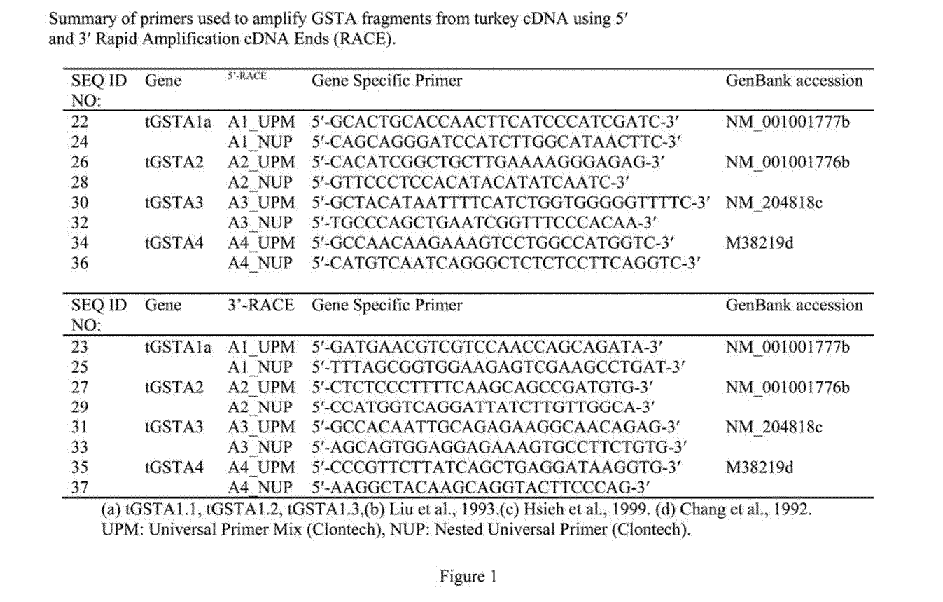

Male day-old turkey poults (Nicholas commercial strain) were obtained from Moroni Feed Co. (Moroni, Utah). Freshly isolated turkey livers were stored in RNAlater (Ambion). Samples were homogenized using a Polytron (Brinkman) and mRNA was extracted using Oligotex Direct mRNA kit (QIAgen). The first strand cDNA was synthesized using MMLV reverse transcriptase (Clontech) and each 5′-CDS primer and 3′-CDS primer provided in SMART™ RACE cDNA Amplification Kit (Clontech), respectively to carry out RACE. Gene-specific primers (FIG. 1) were designed based on sequences of chicken GST α-class transcripts, cGSTA1, cGSTA2, cGSTA3 and cGSTA-CL3 to perform 5′- and 3′-RACE. Fragments were amplified in PCR reactions with both universal primer mix (UPM, long primer: 5′-CTAATACGACTCACTATAGGGCAAGCAGTGGTATCAACGCAGAGT-3′ (SEQ ID NO: 7), and short primer: 5′-CTAATACGACTCACTATAGG GC-3′) (SEQ ID NO: 8) and nested universal primers (NUP, 5′-AAGCAGTGGTATCAACGCAGAGT-3′) (SEQ ID NO: 9) provided in SMART™ RACE cDNA Amplification kit (Clontech).

The following PCR profile was performed with Advantage 2 PCR kit (Clontech): 2 minutes at 94° C., followed by 34 cycles for first reaction and 25 cycles for nested PCR consisting of 30 seconds at 94° C., 30 seconds at an optimal annealing temperature 58-68° C., and 8 minutes extension time at 72° C. PCR products were subcloned in TA cloning vector pDrive (Qiagen) and transformed into chemically competent E. coli, DH5α (Invitrogen). The presence of RACE fragments within the clones was confirmed by colony PCR and sequence analysis. For confirmation of GST gene coding regions, PCR was performed using the proofreading enzyme ultra pfu DNA polymerase (Stratagene) with gene-specific primers (Table 1) and cloning with Zero Blunt PCR11 vector (Invitrogen).

TABLE 1
Summary of Primers used to amplify the open reading frame of α-class
GST fragments from turkey cDNA.

| | | Gene | | | |
|---|---|---|---|---|---|
| Sequence | | Size | | | Genbank |
| ID NO: | Gene | (bp) | Name | Gene specific primers | accession |
| 38 | tGSTA1.1 | 663 | A1.1_F | 5′-ATGTCTGGGAAGCCAGTTCTG-3′ | GQ228399 |
| 39 | | | A1.1_R | 5′-TCA ATGGAAAATTGCCATCA-3′ | |
| 40 | tGSTA1.2 | 666 | A1.2_F | 5′-ATGTCTGGGAAGCCAGTTCTG-3′ | GQ228400 |
| 41 | | | A1.2_R | 5′-TCAGTGGAAAATTGCTATCACACT-3′ | |
| 42 | tGSTA1.3 | 666 | A1.3_F | 5′-ATGTCTGGGAAGCCAGTTCT-3′ | GQ228401 |
| 43 | | | A1.3_R | 5′-TCAACTGAAAATTGCCAGCAG-3′ | |
| 44 | tGSTA2 | 669 | A2_F | 5′ATGGCGGAGAAACCTAAGCTTCACTATACCA-3′ | GQ228402 |
| 45 | | | A2_R | 5′TAATGTGAGGAAAATATTCAGTTTCTAAGGCCGC- | |
| | | | | 3′ | |
| 46 | tGSTA3 | 672 | A3_F | 5′-ATGTCGGAGAAGCCCAGGCTCACCTA-3′ | GQ228403 |
| 47 | | | A3_R | 5′TCAGTCTAGCTTAAAAATTTTCATCACAGTTGC-3′ | |
| 48 | tGSTA4 | 690 | A4_F | 5′-ATGGCTGCAAAACCTGTACTCTACTAC-3′ | GQ228404 |
| 49 | | | A4_R | 5′-CTAATTTGGTTTTACATCATAATACATCCGG-3′ | |

The PCR profile for this reaction with Advantage 2 PCR kit (Clontech) was: 2 minutes at 94° C., followed by 25 cycles consisting of 30 seconds at 94° C., 30 seconds at an optimal annealing temperature at or between 56-60° C., and 8 minute extension at 72° C. Clones were confirmed by colony PCR and sequence analysis. All alignments of genes were analyzed with ClustalW.

### BAC Library Screening and Sequencing

Sequences of turkey GST genes were used to design probes to screen the CHORI-260 BAC library array by overgo hybridization. Overgo sequences were as follows: GSTA2 O2-CAGAGTAGAATTACATTACGTTGCTG (SEQ ID NO: 11); and GSTA2 O1-CTGTATTACTGTTCTGCAGTTACCCA (SEQ ID NO: 10; GSTA3 O1-CAGGCAGATGTAAGAGGAGCACTTC (SEQ ID NO: 12); and GSTA3 O2-TTAGAAGACTTCATTGCGTGGAAGT (SEQ ID NO: 13). Positive BAC clones were identified and grown overnight in LB media containing 25 μg/mL chloramphenicol. BAC DNA was prepared using QIAprep columns (Qiagen) and presence of GSTs was confirmed by PCR (Table 2A). Two GST-positive clones were identified (37H 15 and 08C04) and end sequenced with vector-specific primers (Genbank accession nos. F1907948, F1907949, F1907950, F1907951). Based on the position of the end sequences in the chicken genome, 37H 15 was chosen for full sequencing. BAC DNA was prepared using a large construct kit (Qiagen) and submitted to the Advanced Genetic Analysis Center, University of Minnesota for sublibrary construction and sequencing with Roche 454 GS FLX technology.

TABLE 2
PCR primers used to verify presence of GST
genes within BAC clones prior to sequencing
(A) and for amplification of A 1 gene
regions in BAC assembly (B).

| | | SEQ |
|---|---|---|
| | | ID |
| Name | Sequence | NO: |
| (A) | | |
| GSTA1_prof | 5′-TTAATTAATAATCAGCTGCTTTGC-3′ | 50 |
| GSTA1_pro | 5′-GCTAGCAGCCAGCGTACTG-3′ | 51 |
| GSTA1 2-3f | 5′-ATGGATCCCTGCTGTTCCAG-3′ | 52 |
| GSTA1 2-3r | 5′-CAAAGACTGGGAAATATCTGTTTG-3′ | 53 |
| GSTA1 3-4r | 5′-GCATCAGGCTTCGACTCTTC-3′ | 54 |
| GSTA1 3-4f | 5′-GACATGTATGTGGAAGGACTGG-3′ | 55 |
| GSTA2_int2f | 5′-CTTCAGCTGCCAGGTTTG-3′ | 56 |
| GSTA2_int2r | 5′-AACCCCAGCTGCTGCTAAC-3′ | 57 |
| GSTA3_prof | 5′-GCGTTATGCAAAGCAGAGC-3′ | 58 |
| GSTA3_pror | 5′-AGCGGATCGACTCCATTTTG-3′ | 59 |
| (B) | | |
| A1.1_2f | 5′-CTGCACTATCCCAACTCACG-3′ | 60 |
| A1.2_2f | 5′-CTGCACTATGCTAATATACG-3′ | 61 |
| A1.3_2f | 5′-CTGCACTATGTCAGTGTACG-3′ | 62 |
| A1.1_2r | 5′-ATTCGGCCTCGTGAGTTGGG-3′ | 63 |
| A1.2_2r | 5′-GGTTCCATTCGGCCTCGTATATTAGC-3′ | 64 |
| A1.3_2r | 5′-ATTCGGCCGCGTACACTGAC-3′ | 65 |
| A1.1_3r | 5′-TGTAACTTTTGGAGATCTTCCTTTTT-3′ | 66 |
| A1.2_3r | 5′-TTTGGAGATCATCCTTAGTTTTCAG-3′ | 67 |
| A1.3_3r | 5′-TGGAGATCTTCTTTTGTTTCCAG-3′ | 68 |
| A1_univ_4r | 5′-GGTTGTATTTCCCTGCGATG-3′ | 69 |
| A1_univ_5f | 5′-GTATGTGGAAGGAWTGGCAG-3′ | 70 |
| Al.1_6f | 5′-CCATTTTAGTGGTGGAAGAGC-3′ | 71 |
| A1.2_6f | 5′-TTTTGGGGTTGGAAGAGTTG-3′ | 72 |
| A1.3_6f | 5′-TTTAATGGTGGAAGAGTTCAAGC-3′ | 73 |
| Al.1_7f | 5′-GCCAGAGGAAACCACCTTTAC-3′ | 74 |
| A1.2_7f | 5′-GCGCAAAGAAACCACTGATTC-3′ | 75 |
| A1.3_7f | 5′-GCCCAAGGAAACCACCTCTAC-3′ | 76 |
| A1.1_7r | 5′-GGAAAATTGCCATCAGACTTG-3′ | 77 |
| A1.2_7r | 5′-GGAAAATTGCTATCACACTTG-3′ | 78 |
| A1.3_7r | 5′-TGAAAATTGCCAGCAGGTTTG-3′ | 79 |

### BAC Contig Assembly

Approximately 16,000 454 sequence reads (˜12×) were generated. Sequences were initially assembled with GS Assembler (Roche) and Sequencher software (Gene Codes, Corp) with the tGST cDNAs used to aid alignments. Assembled contigs were confirmed by overlapping gene-specific polymerase chain reaction (PCR) and resequencing using primers anchored within exons (Table 2B). PCR reactions were performed with BAC DNA as template using Hotstar Taq polymerase (Qiagen). Amplifications were performed for 35 cycles with 58° C. annealing temperature and 1 minute/kb extension times. PCR products were purified with PCR cleanup columns (Qiagen) and directly sequenced. Due to the duplicated nature of the three A1 genes, additional large-insert clones were constructed to aid in assembly of the intergenic regions. Gene-specific primers anchored in exons 7 and 2 of adjacent genes were designed to bridge the sequences between the three A1 genes and the A3/A1 and A1/A2 spacers. PCR reactions were performed with BAC DNA as template using Long Range Taq Mastermix (Qiagen). Amplifications were performed for 35 cycles with 62° C. annealing temperature and 1 minute/kb extension times. PCR products were either TA cloned using the pDrive vector (Qiagen) or digested with restriction endonucleases, ligated into a compatibly prepared vector (pBluescript KS+, Stratagene), and transformed into chemically competent DH5α cells (Invitrogen). Plasmids were purified with Qiagen plasmid minipreps and sequenced using vector-specific and internal primers.

### Gene Identification and Annotation

The assembled sequence was analyzed with Softberry FGENESH and the basic local alignment search tool (BLAST) to identify putative transcripts and homologies to known genes. Comparisons between predicted gene sequences, available ESTs, and the published chicken whole genome sequence were performed using Sequencher software (Gene Codes, Corp). Repetitive elements were identified using REPEATMASKER and Tandem Repeats Finder. GC content analysis was performed with 100 bp windows using Isochore. CpG islands were identified with the Softberry CpGfinder using default settings.

### Genetic Mapping of α-Class GST Gene Cluster

In order to confirm position of the turkey α-class GST gene cluster on chromosome MGA2 as predicted by comparative sequence alignments, primers were designed to amplify GST introns to identify segregating polymorphisms in the University of Minnesota/Nicholas Turkey Breeding Farms (UMN/NTBF) mapping population. Targeted regions included intron 2 of tGSTA4, intron 4 of tGSTA 1 (A1.1), and introns 2 and 4 of tGSTA2. In chicken and quail, intron 2 of GSTA4 contains a microsatellite repeat (quail locus GUJ0099). Sequencing of the F1 individuals from the UMN/NTBF mapping population found a polymorphic tetranucleotide repeat (TATC)N also occurs within this intron in the turkey. Primers were designed for PCR amplification (Forward 5′-AAGTTTCCCCAGGCAG-3′ (SEQ ID NO: 14) and Reverse 5′-CACACACTGTATCATACTGGAATTTAC-3′ (SEQ ID NO: 15) and the locus was genotyped. SNPs were identified in intron 4 of tGSTA1.1 (C/T) and in intron 4 of tGSTA2 (C/T) by resequencing. The SNP markers were genotyped (PCR/RFLP) across the UMN/NTBF mapping families by digestion of the PCR amplicons directly with restriction endonucleases (Dde I for GSTA1.1 and Bsp HI for GSTA2) followed by electrophoresis in 1% agarose and manual scoring of alleles. Two-point linkage analysis was performed in combination with previously genotyped markers using Locusmap software.

The following disclosure pertains to various embodiments of the invention and may be useful independently, in combinations thereof, and as a whole. According to the present invention, turkey α-class GST genes were amplified. The full length cDNAs of six α-class GST genes were isolated and amplified from turkey liver using 5′- and 3′-RACE. Availability of the extensive chicken EST database which is genetically close to turkey enabled the design of gene-specific primers to amplify turkey α-class GST genes (FIG. 1 and Table 1). Primers for cGSTA 1 amplified the three related A1 genes, tGSTA 1.1, tGSTA1.2 and tGSTA1.3. Predicted open reading frames (tGSTA1.1, 743 bp with ORF 663 bp; tGSTA 1.2, 724 bp with ORF 666 bp; tGSTA1.3, 666 bp with ORF 666 bp; tGSTA2, 840 bp with ORF 669 bp; tGSTA3, 851 bp with ORF 672 bp; and tGSTA4, 845 bp with ORF 690 bp), were confirmed by PCR amplification with proofreading enzyme followed by DNA sequencing (Table 2). The resulting cDNA sequences are accessioned in GenBank (accession nos. GQ228399, GQ228400, GQ228401 GQ228402, GQ228403 and GQ228404, (Table 1). The open reading frame of tGSTA1.1 (663 bp) lacks three nucleic acids compared to tGSTA 1.2 (666 bp), tGSTA 1.3 (666 bp) and chicken GSTA 1 (666 bp). Significance of the missing three nucleic acids (single codon) is not yet known.

The GSTA gene cluster was physically mapped within a single BAC clone (FIG. 2). Since only a few ESTs corresponding to the α-class tGSTs are accessioned in Genbank databases, there is little information regarding the relative expression patterns of the individual genes in turkeys. However, examination of chicken ESTs shows differential presence of individual transcripts suggestive of expression differences. Although only a single GSTA1 sequence has been described in the chicken, EST evidence indicates presence of three A1-like transcripts as seen in the turkey. Clustering of chicken EST sequences revealed the following proportions: cGSTA 1.1, 23.5%: cGSTA 1.2, 21.4%: cGSTA1.3, 13.9%: cGSTA2, 24.6%: cGSTA3, 13.4%: and cGSTA-CL3 (A4), 3.2%; suggesting that A1.1, A 1.2, and A2 have a 2-fold higher expression than A 1.3 and A3 with A-CL3 (A4) representing a minor GST component.

In another embodiment, there is BAC contig assembly. Because of the low quality assembly and annotation of the α-class GST cluster in the chicken genome, the turkey GST genes could not be correctly aligned, necessitating identification and sequencing of a turkey GST BAC clone. Approximately 16,000 454 sequence reads were generated from the CHORI-260 BAC clone 37H 15. Removal of pTARBAC2.1 vector sequence left a 222,565 bp insert of approximately 12× coverage (FIG. 2A). The BAC insert ended in the terminal exon of the gamma-glutamylcysteine synthetase gene (GCLC) and the fourth exon of transmembrane protein 14A (TMEM 14A).

Several repetitive DNA types were identified in the GST BAC comprising almost 24 kb of the 222.5 kb sequence. These included numerous CR1/LTR and simple sequence repeats. Two large complex repeats were identified including an approximately 450 bp GGAAA/GGCAA pentameric repeat (SEQ ID NO: 16) located at 9.6 kb and a second repeat (CATTTTTTCTTTTTTTTTT) (SEQ ID NO: 17) of approximately 650 bp located at 135.9 kb. The turkey GST BAC had an overall GC content of 42.4%. Notable spikes occurred at 31.5 and 122 kb where GC content exceeded 80% (FIG. 2B). Additional GC spikes are associated with the α-class GSTs. These regions of high GC content correspond to eight CpG islands detected with CpGfinder. A single non-coding 7SK snRNA sequence was found at 145.2 kb. This non-coding RNA appears to be widely conserved in vertebrates being present in the human genome as well as the chicken. The final assembly was annotated and submitted to Genbank (accession no: GQ254850).

In yet another embodiment are genes and gene identification and annotation. Based on BLAST homologies, FGENESH, and EST analysis, the 222.5 kb region contained 15 predicted genes (FIG. 2A). These include the six α-class GSTs (tGSTA1.1, tGSTA 1.2, tGSTA 1.3, tGSTA2, tGSTA3, and tGSTA4) identified by RACE, the ELOVL5 (Elongation of very long chain fatty acids) homolog, GCM (glial cells missing homolog 1), FBX09 (F-box protein 9), ICK (intestinal cell (MAK-like) kinase), a sequence similar to transcribed locus B1335628, and two predicted genes not supported by EST data. Partial coding sequences are also included for GCLC and THEM14A as previously discussed. Single amino acid differences were observed between the tGSTA2, tGSTA3, and tGSA4 ORFs in the BAC clone versus those amplified by RACE. These differences can be attributed to the different DNA sources used for RACE amplification (Nicholas commercial strain) and the BAC clone.

In yet another embodiment, there is genetic mapping of GST α-class. To verify position of the α-class GST in the turkey genome, a polymorphic tetranucleotide repeat (tGUJ099) within intron 2 of tGSTA4 (cGSTCL3) and SNPs within tGSTA1.1 and tGSTA2 were genotyped across the UMN/NTBF mapping families. The number of informative meioses was 171 for tGUJ099, 168 for the tGSTA 1.1 SNP, and 124 for tGSTA2 SNP. Significant genetic linkage (LOD>3.0) was observed between tGUJ099 and ten previously linked loci on MGA2 (Table 3).

TABLE 3
Pairwise linkage analysis of GST markers
in the UMN/NTBF mapping population.

| GST locus | Linked loci | Theta | LOD |
|---|---|---|---|
| tGUJ099 | GSTA1.1SNP | 0.025 | 37.66 |
| | GSTA2SNP | 0.097 | 18.23 |
| | MNT045 | 0.349 | 4.76 |
| | MNT070 | 0.388 | 3.01 |
| | MNT080 | 0.238 | 10.3 |
| | MNT155 | 0.159 | 4.87 |
| | MNT217 | 0.022 | 11.75 |
| | MNT329 | 0.284 | 10.3 |
| | MNT379 | 0.378 | 3.00 |
| | MNT382 | 0.023 | 10.88 |
| | MNT415 | 0.064 | 15.83 |
| | RHT259 | 0.185 | 19.68 |
| GSTA1.1SNP | GSTA2SNP | 0.085 | 19.53 |
| | MNT045 | 0.362 | 4.17 |
| | MNT080 | 0.238 | 10.59 |
| | MNT155 | 0.105 | 5.88 |
| | MNT217 | 0.043 | 10.27 |
| | MNT329 | 0.266 | 12.62 |
| | MNT382 | 0.047 | 9.43 |
| | MNT415 | 0.093 | 13.11 |
| | RHT259 | 0.195 | 18.26 |
| GSTA2SNP | MNT080 | 0.311 | 4.01 |
| | MNT217 | 0.135 | 4.77 |
| | MNT329 | 0.314 | 5.09 |
| | MNT382 | 0.088 | 5.83 |
| | MNT415 | 0.156 | 6.37 |
| | RHT259 | 0.234 | 10.00 |

As expected, this microsatellite was also linked to both GST SNPs and the SNPs were linked to each other (θ=0.085, LOD=19.53) and to a subset of the loci found linked to tGUJ099. These results place the turkey GST genes on the distal q arm of chromosome 2 (MGA2) which is homologous to chicken chromosome 3 (GGA3).

### GST Nomenclature

Nomenclature for turkey cytosolic GSTs should reflect primary structural similarities, the division of GSTs into classes of more closely related sequences and the position of the α-class GST genes within the sequence cluster as aligned with the human genome sequence (conserved synteny, FIG. 2C). Amino acid similarities among the tGSTs ranged from 62 to 86% with an average of 70%. This is similar to that observed in chicken where similarities range from 64 to 71% (average 68%, Table 4) and comparable values are seen among human GSTAs (Table 5).

TABLE 4
Amino acid sequence similarities among turkey and chicken α-class GSTs.

| | | | | | | | | | |
|---|---|---|---|---|---|---|---|---|---|
| | | | | | | | | | cGSTA- |
| tGSTA1.1 | tGSTA1.2 | tGSTA1.3 | tGSTA2 | tGSTA3 | tGSTA4 | cGSTA1 | cGSTA2 | cGSTA3 | CL3 |

| | | | | | | | | | |
|---|---|---|---|---|---|---|---|---|---|
| tGSTA1.1 | 80 | 83 | 70 | 65 | 62 | 91 | 70 | 66 | 63 |
| tGSTA1.2 | | 86 | 69 | 65 | 63 | 81 | 69 | 63 | 63 |
| tGSTA1.3 | | | 71 | 66 | 64 | 82 | 69 | 63 | 63 |
| tGSTA2 | | | | 68 | 70 | 71 | 99 | 68 | 71 |
| tGSTA3 | | | | | 68 | 66 | 68 | 94 | 69 |
| tGSTA4 | | | | | | 63 | 70 | 67 | 97 |
| cGSTA1 | | | | | | | 71 | 66 | 64 |
| cGSTA2 | | | | | | | | 68 | 70 |
| cGSTA3 | | | | | | | | | 69 |
| cGSTA-CL3 | | | | | | | | | |

Turkey and chicken GSTs are on average 95% similar in amino acid sequence (Table 4) indicating these represent orthologues (only a single GSTA 1 gene has been formally described in the chicken although three genes were identified in accessioned ESTs). Given the observed synteny between the human and turkey α-class gene cluster and flanking genes, we have designated three of the turkey α-class genes as follows: tGSTA4 (=hGSTA4 and cGST-CL3), tGSTA3 (=hGSTA3 and cGSTA3) and tGSTA2 (=hGSTA2 and cGSTA2). Nomenclature of the remaining “tGSTA1-like” genes is problematic given their close DNA sequence and amino acid similarities. We have provisionally designated the three tGSTA1-like genes as tGSTA1.1, tGSTA1.2 and tGSTA1.3 pending future functional assays. Highest amino acid similarity is seen among the three tGSTA1 genes (average 83%) with GSTA1.2 and GSTA1.3 being the most similar (86%, Table 4). When compared to the human GST sequences the tGSTA1 genes are slightly more similar to hGSTA1 (average 63.6% similarity) than to other human α-class GST genes: hGSTA2 (62.6%), hGSTA3 (62.6%), hGSTA4 (59%) and hGSTA5 (61.3%) (Table 5).

TABLE 5
Amino acid sequence similarities among
turkey and human α-class GSTs.

| | hGSTA1 | hGSTA2 | hGSTA3 | hGSTA4 | hGSTA5 |
|---|---|---|---|---|---|
| tGSTA1.1 | 65 | 63 | 64 | 61 | 63 |
| tGSTA1.2 | 62 | 62 | 61 | 58 | 60 |
| tGSTA1.3 | 64 | 63 | 63 | 58 | 61 |
| tGSTA2 | 69 | 69 | 68 | 59 | 69 |
| tGSTA3 | 64 | 64 | 63 | 61 | 62 |
| tGSTA4 | 62 | 62 | 62 | 61 | 62 |
| hGSTA1 | | 95 | 90 | 53 | 90 |
| hGSTA2 | | | 88 | 54 | 88 |
| hGSTA3 | | | | 53 | 85 |
| hGSTA4 | | | | | 53 |

The degree of shared synteny between turkey and human GST orthologues is interesting given the estimated divergence of the avian lineage from mammals 300-350 million years ago (Mya). Presence of orthologous genes on homologous chromosome segments (conserved synteny) reflects both common phylogenetic origin and ancestral genomic organization. Examples of this are seen on human chromosome 9 (HSA9) where homologs for 11 of 18 genes found on the chicken Z chromosome are located (Nanda et al., 1999; Nanda et al., 2000), and the GST locus where α-class GSTs and flanking genes are shared between human (HSA6) and turkey (MGA2). While conservation of gene order indicates ancestral genomic organization, conservation of gene clusters is indicative of local gene duplications that occurred early in vertebrate evolution. One example is the melanocortin receptors (MC2R, MC5R, and MC4R) in chicken (GGA2), humans (HSA18), and other mammals where gene clusters appear to reflect ancient local gene duplication (Schioth et al., 2003). The turkey α-class GST cluster appears to contain both genes of ancient local duplication (GSTA2-4) and others of more recent events (GSTA.1, A1.2 and A1.3).

In yet another embodiment of the present disclosure, turkey α-class GST domains are provided. The alignment of six α-class GST proteins demonstrated that all six genes encode α-class tGST proteins based on two conserved domains and four signature motifs (FIG. 3). PROSITE and BLOCKS queries for conserved domains of α-class of tGSTs revealed the presence of an N-terminal thioredoxin conserved domain (3-83 amino acids) and a C-terminal conserved α-helical domain (tGSTA1.1, tGSTA1.2 and tGSTA4: 85-207aa: tGSTA1.3 and tGSTA2: 85-208 aa; tGSTA3: 85-209aa). PANTHER and BLOCK searches revealed four putative motifs predicting high catalytic activity toward cumene hydroperoxide and 7-chloro-4-nitrobenz-2-oxa-1,3-diazole (NBD), amongst other substrates. In addition, α-class tGSTs exhibit a number of differences from the characteristic GST structure (Dirr et al., 1994). The signature motifs and conserved residues of α-class tGST proteins are divided into four motifs with conserved residues: (1) 15-29 aa (xMExxxWLLAAAGVE) (SEQ ID NO: 18), (2) 82-98 aa (YGKDxKERAxIDMYVxG) (SEQ ID NO: 19), (3) 134-148 aa (PVxEKVLKxHGxxxL) (SEQ ID NO: 20) and (4) 191-208 aa (PxIKKFLxPGSxxKPxxx) (SEQ ID NO: 21).

Interestingly, tGSTA1.1 contains only three motifs (1, 2 and 4) (FIG. 3) due to lack of one amino acid (position 142) within the signature motif. This gene (220 aa) has 91% sequence similarity with the single described cGSTA1 (221 aa) (Table 4). Since a single point mutation in the hydrophobic substrate-binding site region is enough to shift substrate specificity from class π to α, it is possible that these differences may explain substrate specificities of the turkey forms of GSTA. Turkey liver cytosolic GSTs are active toward prototypical substrates, such as chlorodinitrobenezene (CDNB) and dichlornitrobenzene (DCNB), but not toward the AFB1 metabolite AFBO, a condition posited to be a critical determinant for the extreme sensitivity of turkeys toward AFB1.

### Determination of Liver Cytosolic AFB₁ GST Detoxification Activity

GST mediated conjugation activities toward AFB₁ were accomplished determined in turkey hepatic cytosolic fractions (Klein, Buckner, Kelly and Coulombe, 2000; Klein and Coulombe, 2000; Klein, Van Vleet, Hall, and Coulombe, 2002a; Klein, Van Vleet, Hall, and Coulombe, 2002b; Klein Van Vleet, Hall, and Coulombe 2003). Turkey liver microsomes (˜400 μg total protein) used as a source of AFB1 bioactivation, were mixed and reacted with 100 μM AFB1 (in DMSO), 2 mM NADPH, 5 mM GSH, and turkey hepatic cytosol (˜800 μg total protein) containing GST (Guarisco, Hall, and Coulombe, 2008; Klein, Van Vleet, Hall, and Coulombe, 2003). This mixture was incubated in epoxide trapping buffer (5 mM MgCl₂, 25 mM KCl, 0.25 mM sucrose and 80 mM potassium phosphate, pH 7.6) to give a final volume of 250 μL. The reaction mixture was incubated at 37° C. for 20 minutes with gentle shaking and stopped by adding 250 μL of cold MeOH spiked with 24 μM aflatoxin G₁ as an internal standard. The samples were kept overnight at −20° C. to facilitate protein precipitation and then centrifuged at 13,000 g for 10 minutes, and the supernatants filtered through a 0.2 μM nylon membrane and 100 μL injected into HPLC. Metabolites were separated on an HPLC system and elution of the peaks monitored by UV absorbance (λ=365 nm). Amounts of metabolite formation were calculated by establishing calibration curves between the peak areas in the chromatograms and the amount of metabolite injected, using an authentic exo-AFB₁-GSH standard, synthesized in our laboratory. Representative results are presented in Table 6.

TABLE 6
Comparison of hepatic cytosolic AFB₁ Detoxification
activities in domestic turkey (DT), Eastern Wild (EW),
Rio Grande Wild (RGW), and Royal Palm (RP) Turkeys

| Liver sample | AFB₁ detoxification¹ |
|---|---|
| Domestic turkey | n.d.² |
| EW #6 | 0.100 ± 0.00 |
| EW #7 | 0.099 ± 0.00 |
| EW#10 | 0.080 ± 0.00 |
| RGW #12 | 0.076 ± 0.00 |
| RP #5 | 0.054 ± 0.00 |
| RP #8 | 0.066 ± 0.00 |
| Mouse | 0.426 ± 0.04 |

¹Specific enzyme activity (nmol/min per mg protein); n.d. = not detected. BHA-induced mouse liver, the “gold standard” for AFB detoxification, included for comparison. Mean ± SD of triplicate determination;
²Negative AFB detoxification activity measured in livers of >250 domestic turkeys (Nicholas strain).

### SNP Determination

GST gene fragments (GST and flanking genes) from turkeys included in the GST bioassays were amplified by polymerase chain reaction (PCR) for SNP genotyping. SNPs were genotyped by PCR/RFLP as an efficient means of examining single genomic regions and for determining haplotypes. From the turkey GST sequences, PCR primers were used that amplify specific fragments spanning portions of the GST homologues and genes flanking the GST locus. Selected SNPs were genotyped across all individuals included in the bioassays. Briefly, DNA fragments were amplified by PCR and the products digested with restriction endonucleases selected as containing the target SNP as part of the recognition sequence. A person skilled in the art of genetic screening and molecular biology will easily identify which restriction enzymes will cleave at the desired region that includes a SNP. Examples include HpaII or MspI which cleaves when the SNP is present at nucleotide 402 of tGSTA 1.2 and Dra I which cleaves when the SNP is present at nucleotide 420 of tGSTA 1.2. Neither enzyme is predicted to cleave when an alternate nucleotide is present at these positions in the tGSTA 1.2 DNA sequence. Other examples include Dde I for identifying SNPS in GSTA 1.1 and Bsp HI for identifying SNPS in GSTA2. The digested fragments may then be separated by electrophoresis in agarose. The size of the DNA fragments indicate if the restriction enzyme cut the PCR fragment and, consequently, if the SNP sequence is present in the PCR fragment. Fragments were then separated by electrophoresis in agarose and manually scored, or size fractionated on the QIAxcel multicapillary electrophoresis system. Polymorphisms were analyzed using PHASE and Haploview software (Barrett, Fry, Maller, and Daly, 2005; Stephens and Donnelly, 2003) to impute SNP GST haplotypes. We have successfully employed this technique to determine MHC SNP haplotypes in divergent genetic backgrounds of commercial and wild turkeys (Chaves, Harry, and Reed, 2009; Chaves, Krueth, Bauer, and Reed, 2011). It will be appreciated by one of skill in the art of genetic screening and molecular biology that other laboratory techniques such as the kinetic PCR technique (Germer, Holland, and Higuchi, 2000) or other molecular biology techniques may be used to identify the presence or absence of the SNPs described herein.

TABLE 7
Single Nucleotide Polymorphisms (SNPs) of six Alpha-class genes
in wild and domestic turkeys

| | No | | | |
|---|---|---|---|---|
| GST | of | | | |
| isoform | SNPs | Enzyme | Position (bp) | Position (aa) |
| tGSTA1.1 | 5 | | 34 72 146 327 569 | 12 24 49 109 190 |
| (663 bp) | | A1.1¹ | T A C C C | Ser Ala Ser Asn Thr |
| | | EWA1.1² | A G T T T | Thr Ala Phe Asn Ile |
| | | RGWA1.1³ | T A C C C | Ser Ala Ser Asn Thr |
| | | RPA1.1⁴ | A G T T T | Thr Ala Phe Asn Ile |
| tGSTA1.2 | 7 | | 146 243 402 420 432 496 606 | 49 81 134 140 133 166 202 |
| (666 bp) | | A1.2 | G C A G G G C | Cys Leu Pro Leu Gly Gly Ser |
| | | EWA1.2 | G T A G G G T | Cys Leu Pro Leu Gly Gly Ser |
| | | RGWA1.2 | C T G C A T C | Ser Leu Pro Phe Gly Trp Ser |
| | | RPA1.2 | G T A G A T T | Cys Leu Pro Leu Gly Trp Ser |
| tGSTA1.3 | 3 | | 487 514 558 | 163 172 186 |
| (666 bp) | | A1.3 | T C C | Thr Pro Ala |
| | | EWA1.3 | C T A | Thr Ser Ala |
| | | RGWA1.3 | C T A | Thr Ser Ala |
| | | RPA1.3 | C T A | Thr Ser Ala |
| tGSTA2 | 3 | | 204 255 636 | 68 85 212 |
| (669 bp) | | A2 | T T G | Thr Asp Ser |
| | | EWA2 | T T A | Thr Asp Ser |
| | | RGWA2 | C C A | Thr Asp Ser |
| | | RPA2 | T T A | Thr Asp Ser |
| tGSTA4 | 2 | | 219 507 | 73 169 |
| (690 bp) | | A4 | C A | Asn Glu |
| | | EWA4 | T G | Asn Glu |
| | | RGWA4 | C G | Asn Glu |
| | | RPA4 | C A | Asn Glu |

¹Domestic turkey - A 1-A4;
²EWA = Eastern Wild turkey;
³RGW = Rio Grande Wild turkey;
⁴Royal Palm “heirloom”

Association between individual SNP haplotypes (and/or individual SNPs) and GST activity were then modeled and tested by Chi-square contingency test. Briefly, GST activities measured from livers were treated as a continuous variable. These were compared statistically among haplotypes to see if particular haplotypes (or individual SNPs) are associated with higher GST activity. Groups were compared using p<0.05 considered as statistically significant. Representative results are presented in Table 7.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

1. A method for determining aflatoxin susceptibility or resistance in a subject organism comprising the steps of:
a. obtaining a sample from a subject organism, wherein the sample contains genomic DNA or RNA;
b. screening the sample for the presence of single nucleotide polymorphisms (SNPs) in an α-class glutathione S-transferase (GSTA) gene in the genomic DNA or RNA of the sample;
c. comparing the identified SNPs to sequences in a signature motif within a GSTA nucleotide sequence associated with aflatoxin resistance; wherein presence of at least one SNP in the GSTA gene indicates aflatoxin resistance, and absence of all SNPs indicates aflatoxin susceptibility.

2. The method of claim 1 wherein the GSTA genes comprise any of GSTA1.1 (SEQ ID NO: 1), GSTA1.2 (SEQ ID NO: 2), GSTA 1.3 (SEQ ID NO: 3), GSTA2 (SEQ ID NO: 4), GSTA3 (SEQ ID NO: 5), or GSTA4 (SEQ ID NO: 6).

3. The method of claim 2 wherein the subject organism is poultry.

4. The method of claim 3, wherein the subject organism is turkeys.

5. The method of claim 3, wherein the subject organism is chickens.

6. The method of claim 1 wherein the SNPs comprise any one of:
a. A₃₄, G₇₂, T₁₄₆, T₃₂₇, or T₅₆₉ in the GSTA1.1 nucleotide sequence (SEQ ID NO: 1), C₁₄₆, T₂₄₃, G₄₀₂, C₄₂₀, A₄₃₂, T₄₉₆ or T₆₀₆ of the GSTA1.2 nucleotide sequence (SEQ ID NO: 2), C₄₈₇, T₅₁₄, or A₅₅₈ of the GTSA1.3 nucleotide sequence (SEQ ID NO: 3), C₂₀₄, C₂₅₅, or A₆₃₆ of the GSTA2 nucleotide sequence (SEQ ID NO: 4), or T₂₁₉ or G₅₀₇ of the GSTA4 nucleotide sequence (SEQ ID NO: 6).

7. An isolated polynucleotide comprising at least 9 consecutive bases to about 100 consecutive bases of any one of SEQ ID NOS: 1-6 or the complements thereof, wherein said isolated polynucleotide includes at least one nucleotide selected from a group consisting of SNPs at positions A₃₄, G₇₂, T₁₄₆, T₃₂₇, or T₅₆₉ in the GSTA1.1 nucleotide sequence (SEQ ID NO: 1), C₁₄₆, T₂₄₃, G₄₀₂, C₄₂₀, A₄₃₂, T₄₉₆ or T₆₀₆ of the GSTA1.2 nucleotide sequence (SEQ ID NO: 2), C₄₈₇, T₅₁₄, or A₅₅₈ of the GTSA1.3 nucleotide sequence (SEQ ID NO: 3), C₂₀₄, C₂₅₅, or A₆₃₆ of the GSTA2 nucleotide sequence (SEQ ID NO: 4), or T₂₁₉ or G₅₀₇ of the GSTA4 nucleotide sequence (SEQ ID NO: 6).

8. An isolated polynucleotide of claim 7 wherein the polynucleotide is labeled.

9. A kit comprising at least one isolated polynucleotide of claim 7.

10. A kit of claim 9 further comprising instructions for the use thereof.

11. A kit of claim 9 wherein at least one of the polynucleotides is labeled.

12. An array comprising a plurality of polynucleotides immobilized on a substrate, wherein said plurality comprises the isolated polynucleotide of claim 7.

13. A kit comprising anti-peptide antibodies which bind to peptides comprising one or more of:
a. Thr₁₂, Phe₄₉, or Ile₁₉₀ of the GSTA1.1 peptide (SEQ ID NO: 80), Ser₄₉, Phe₁₄₀, or Trp₁₆₆ of the GSTA1.2 peptide (SEQ ID NO: 81), or Ser₁₇₂ of GSTA1.3 peptide (SEQ ID NO: 82).

14. An array comprising a plurality of anti-peptide antibodies immobilized on a substrate wherein said plurality comprises the anti-peptide antibodies of claim 13.

15. A method for generating aflatoxin resistance in poultry comprising:
a. screening poultry for SNPs in the nucleotide sequences for GSTA genes;
b. selecting birds which possess the GSTA nucleotide sequences associated with aflatoxin resistance as founder birds for breeding;
c. breeding said founder birds;
d. screening the offspring from said founder birds for GSTA SNPs to identify birds homozygous for GSTA sequence associated with aflatoxin resistance; and
e. cross-breeding birds found to be homozygous for GSTA sequence associated with aflatoxin resistance to create an aflatoxin resistant flock.

16. The method of claim 15 wherein the poultry is turkeys.

17. The method of claim 15 wherein the poultry is chickens.
